(19) United States
(12) Patent Application Publication
(10) Pub. No.: US 2016/0220611 A1
(43) Pub. Date: Aug. 4, 2016

(54) Ischemic Tolerant Cells and Cellular Factors in the Treatment of Acute Coronary Syndrome

(71) Applicant: Stemedica Cell Technologies, Inc., San Diego, CA (US)

(72) Inventors: A. U. Djolbasbekova, Astana (KZ); Daniyar Djumaniyazov, Astana (KZ)

(21) Appl. No.: 14/608,470

(22) Filed: Jan. 29, 2015

(51) Int. Cl. A61K 35/28; A61K 9/00

(52) U.S. Cl. CPC A61K 35/28; A61K 9/0019; A61K 2035/124

(57) ABSTRACT

A method for treatment of coronary conditions comprising administering an effective amount of therapeutic stem cells. Included within the scope of the invention is the intravenous administration of a therapeutic amount of ischemic tolerant mesenchymal stem cells in the treatment of acute coronary syndrome.

The present application derives benefit of U.S. Provisional Application 61/915,495 filed Dec. 12, 2013.

### FIELD OF THE INVENTION

The invention relates to the use of stem cells in the treatment of coronary conditions. More particularly, the invention relates to a method of using ischemic tolerant cells, and cellular factors derived therefrom, in the treatment of heart conditions such as acute coronary syndrome.

### SUMMARY OF THE INVENTION

An objective of the invention is to provide a method for the treatment of acute coronary syndrome in a patient comprising administering a therapeutically effective amount of ischemic tolerant mesenchymal stem cells.

### DEFINITIONS

As used herein, the term “stem cell” refers to an undifferentiated cell which has the ability to both self-renew (through mitotic cell division) and undergo differentiation to form a more specialized cell. Stem cells have varying degrees of potency. A precursor cell is but one example of a stem cell.

As used herein, the term “mesenchymal cell” refers to mesodermal germ lineage cells which may or may not be differentiated. The mesenchymal cells of the invention include cells at all stages of differentiation beginning with multipotent mesenchymal stem cells, down to fully differentiated terminal cells.

As used herein, the term “ischemic tolerant” may be used to describe a cell, cell culture or tissue which has been exposed to atmospheric conditions having an oxygen concentration that is less than ambient air. Such exposure may include, but is in no way limited to, priming cells and/or growing cells under low oxygen conditions.

As used herein, the term “patient,” or “subject,” refers to animals, including mammals, preferably humans, who are treated with the pharmaceutical compositions or accordance with the methods described herein

As used herein, the term “pharmaceutically acceptable carrier” (or medium), which may be used interchangeably with the term “biologically compatible carrier” (or medium), refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

As used herein, the term a “therapeutically effective amount,” or “effective amount,” refers to the amount of a composition that produces a therapeutic effect or improvement in a targeted disorder.

As used herein, the term “stem cell composition” refers to a composition containing whole stem cells, stem cell factors, microvesicles, or a combination thereof.

As used herein, the term “treating,” or “treat,” refers to producing a therapeutic effect in a targeted condition through the administration of an effective amount of a composition. Such therapeutic effects include preventing a pathologic condition from occurring, inhibiting the pathologic condition or arresting its development, relieving (e.g. curing or reversing) a pathologic condition, or alleviating the symptoms associated with a pathological condition.

As used herein, the term “clone,” or “clonal cell,” refers to a single cell which is expanded to produce an isolated population of phenotypically similar cells (i.e. a “clonal cell population”).

As used herein, the term “cell line” refers to one or more generations of cells which are derived from a clonal cell.

The terms “administration” and “administering” as used herein refer to the delivery of therapeutic composition by an administration route including, but not limited to, intravenous, intra-arterial, intramuscular, intraperitoneal, subcutaneous, intramuscular, topically, or combinations thereof.

### DETAILED DESCRIPTION

The invention relates to the administration of a composition of cells in the treatment of coronary conditions, such as acute coronary syndrome, for example. In a preferred embodiment, the invention administers mesenchymal stem cells. In still a more preferred embodiment, the invention administers ischemic tolerant stem cells.

Stem cells for use with the invention include mesenchymal stem cells (MSC). Such MSC may be obtained from prenatal sources, postnatal sources, and combinations thereof. Tissues for deriving a suitable MSC include, but are not limited to, bone marrow, blood (peripheral blood), dermis (e.g. dermal papillae), periosteum, synovium, peripheral blood, skin, hair root, muscle, uterine endometrium, adipose, placenta, menstrual discharge, chorionic villus, amniotic fluid and umbilical cord blood. Mesenchymal stem cells may be derived from these sources individually, or the sources may be combined (before or after enrichment) to produce a mixed population of mesenchymal stem cells from different tissue sources.

Mesenchymal stem cell compositions for use with the invention may comprise purified or non-purified mesenchymal stem cells. Mesenchymal stem cells for use with the invention include, but are in no way limited to, those described in the following references, the disclosures of which are incorporated herein by reference: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; U.S. Pat. No. 8,790,638; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

Ischemic tolerant stem cells (e.g. MSC) for use with the invention are grown (i.e. cultured) under low oxygen conditions. Without being limited to any particular theory, culturing the stem cells under low oxygen conditions increases stem cell proliferation and enhances the production of stem cell factors beneficial in the regeneration (and rejuvenation) of tissues in vivo.

The term “low oxygen,” or “low oxygen conditions,” as used herein refers reduced oxygen tension (i.e. any oxygen concentration that is less than atmospheric oxygen). Thus, the stem cells for use with the invention may be grown in an oxygen concentration that is below about 20%, preferably below about 15%, more preferably below about 5-10%, at sea level. Low oxygen conditions may be kept as close as possible to the normal physiological oxygen conditions in which a particular stem cell would be found in vivo.

In one embodiment, the low oxygen conditions comprise an ambient (e.g. incubator) oxygen condition of between about 0.25% to about 18% oxygen. In another embodiment, the ambient oxygen conditions comprise between about 0.5% to about 15% oxygen. In still another embodiment, the low ambient oxygen conditions comprise between about 1% to about 10% oxygen. In further embodiments, the low ambient oxygen conditions comprise between about 1.5% to about 6% oxygen. Of course, these are exemplary ranges of ambient oxygen conditions to be used in culture and it should be understood that those of skill in the art will be able to employ oxygen conditions falling in any of these ranges generally or oxygen conditions between any of these ranges that mimics physiological oxygen conditions for the particular cells. Thus, one of skill in the art could set the oxygen culture conditions at 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, or any other oxygen condition between any of these figures

Methods for manufacturing stem cells under low oxygen conditions as disclosed herein are available in the art, including the methods disclosed in the following publications, the disclosures of which are incorporated herein by reference. U.S. Pat. No. 6,759,242; U.S. Pat. No. 6,846,641; U.S. Pat. No. 6,610,540; U.S. Pat. No. 8,790,638; J. Cereb. Blood Flow Metab. 2008 September 28(9):1530-42; Stem Cells. 2008 May 26(5):1325-36; Exp Neurol. 2008 April 210(2):656-70; Mol. Cell. Neurosci. (2007), doi:10.1016/j.mcn.2007.04.003; Experimental Neurology 170, 317-325 (2001); and Neurosignals 2006-07, 15:259-265. Although these references disclose particular procedures and reagents, any low oxygen culture condition capable of expanding stem cells according to the invention may be used.

As noted above, the invention is practiced by administering a stem cell composition to a patient suffering from a coronary condition. As used herein, the terms “administering,” “administered” and “administer” refer to any administration route by which a stem cell composition can be administered to a patient for a therapeutic effect as disclosed herein. For example, the stem cell composition may be administered intravenously, intra-arterially, intramuscularly, intraperitoneally, subcutaneously, intramuscularly, intranasally, sublingually, or a by combination thereof. In a preferred embodiment, the stem cell composition is administered intravenously.

The term “coronary condition” refers to” to a diagnosis or presumptive diagnosis of cardiovascular disease or conditions affecting the heart that are associated with atherosclerosis, ischemic syndromes, cardiomyopathies, antirythmias, dyserhythmias, hypertension and infections. Coronary conditions include, but are not limited to, acute coronary syndrome (ACS), such as one ST elevation myocardial infarction, non ST elevation myocardial infarction, unstable angina, and chronic heart failure (CHF).

In an exemplary, non-limiting embodiment, the invention is practiced by intravenously administering to a patient a composition of ischemic tolerant mesenchymal stem cells (MSC). MSC for use with this non-limiting embodiment may be derived from human bone marrow grown under low oxygen conditions. The MSC may be grown under low oxygen conditions starting with a primary culture of cells, or passage 2 MSC. Such culture may be maintained under low oxygen conditions for multiple passages, up to the point of harvest for administration to a patient. It is further contemplated in such embodiment that the MSC may be primed with oxygen, or exposed to normoxic oxygen conditions, prior to administration to a patient.

### Example 1—Culture of Low Oxygen Bone Marrow MSC

MSC were derived from the bone marrow of a healthy donor. Mononuclear cells were isolated from a fresh specimen of bone marrow using Histopague and seeded into Petri dishes. The cells were expanded in DMEM/F12 medium containing FGF-2 and 10% fetal bovine serum (FBS). The cells were tested for human pathogens and further expanded up to passage 5 under 5% oxygen conditions. Low oxygen conditions were initiated with passage 2.

### Example 2—Administration of Ischemic Tolerant Cells

The purpose of this study was to determine the effects of ischemic tolerant mesenchymal stem cells (itMSCs) in patients with acute coronary syndrome (STEMI) with left ventricular systolic dysfunction and ejection fraction ≦45%. Human subjects were selected for a study based on the following inclusion/exclusion criteria:

Trial inclusion criteria
Age: less than 60;
STEMI Infarction type in accordance with World Health Organization classification;
Percutaneous coronary intervention (PCI) (coronary angioplasty) performed within 12 hours from the beginning of pain syndrome;
Single-vessel disease with still patent infarct-related artery.
LVEF ≦45% post coronary angioplasty

Trial exclusion criteria
Past incidence of myocardial infarction;
Cardiomyopathy;
Atrial fibrillation or atrial flutter;
Heart surgery in past;
Critical heart valve disorder;
Disorder of hematopoietic system;
Heart insufficiency type IV functional classification of New York Heart Association (NYHA);
Critical renal, lung or liver disorder, or cancer;
Confirmed damage of more than one of three main coronary arteries;
Intracardiac thrombus; bone marrow disorder.

Day 0: Patients with STEMI, undergone successful percutaneous coronary intervention of artery affected by infarction within 12 hours from inciting event.

Day 1-2: Randomization of patients in two groups, ECG, echocardiogram, collection of blood samples after myocardial infarction. 25 patients were selected and grouped as follows: 10 patients were assigned to an experimental group and 15 patients were assigned the control group.

Day 7: The experimental group received an intravenous injection of about 25-100×10⁶ cells ischemic tolerant MSC from Example 1. The control group received an intravenous injection of saline solution.

Day 14 and 3 Months after MSC or PS administration: ECG, echocardiogram, collection of blood samples

6 Months after MSC or PS administration: ECG, echocardiogram, collection of blood samples

1 Year after MSC or PS administration: ECG, echocardiogram, collection of blood samples.

### Example 3 —Results

TABLE 1

| | Experimental | Control | |
|---|---|---|---|
| Patient data | group | group | P |
| Patient number | 19 | 15 | |
| Average age | 51.4 ± 3.5 | 52.7 ± 6.1 | Not-signif |
| Average time for | 5.7 ± 2.5 | 4.5 ± 3.7 | Not signif |
| recanelization of infarct related | | | |
| artery, hours | | | |
| % occlusion of infarct related | 79/14/7 | 71/21/8 | Not signif |
| artery: anterior descending | | | |
| artery (PNA)/right coronary | | | |
| artery (PCA)/circumflex | | | |
| artery, % | | | |
| Degree of acute failure in | 19/45/34/2 | 24/42/35 | Not signif |
| accordance with Killip | | | |
| functional classification | | | |
| I/II/III/IV, % | | | |
| Presence of preinfarction | 46 | 53 | Not signif |
| angina, % | | | |
| Early postinfarction angina, % | 54 | 48 | Not signif |

TABLE 2

| | Experimental group | | Control group | | |
|---|---|---|---|---|---|
| | | 2 | | 4 | |
| | 1 | After 3 | 3 | After 3 | P for |
| Data | Initially | months | Initially | months | 2 and 4 |
| High sensitive C- | 25.3 ± 7.1 | 3.3 ± 1.5* | 28.7 ± 35 | 13.4 ± 7.3* | <0.001 |
| reactive protein, | | | | | |
| mg/ml | | | | | |
| BNP protein, ng/ml | 862.6 ± 123.5 | 119.2 ± 35.7* | 998 ± 113.7 | 1451 ± 212.8 | <0.001 |
| End-diastolic | 146.4 ± 13.3 | 115.9 ± 21.4* | 137.9 ± 33.1 | 143 ± 53.9 | >0.05 |
| volume (EDV) of | | | | | |
| left ventricle (LV), | | | | | |
| ml | | | | | |
| End-systolic volume | 69.2 ± 8.6 | 46.7 ± 6.3* | 66.9 ± 9.1 | 75.6 ± 11.5 | <0.05 |
| (ESV) of LV, ml | | | | | |
| Quantity of | 8.2 ± 2.9 | 2.6 ± 1.1* | 7.9 ± 3.5 | 5.6 ± 2.2 | >0.05 |
| asynergic segments | | | | | |
| of LV | | | | | |
| Ejection fraction of | 42.1 ± 6.1 | 57.5 ± 3.3* | 46.9 ± 7.1 | 45.5 ± 6.7 | <0.05 |
| LV, % | | | | | |
| Functional class of | | 1.5 ± 0.7 | | 3.1 ± 0.3 | <0.05 |
| chronic heart failure | | | | | |
| in accordance with | | | | | |
| (NYHA) | | | | | |

Administration of itMSC resulted in: statistically significant decrease in inflammation as judged by the level of C-reactive protein; in significant decrease in end-systolic and end-diastolic volume of left ventricle, as well as significant increase in the LVEF from 38.4% to 52.3% at three months and to 54.7% at six months post-administration, which brought his parameter to what is considered to be a normal range for healthy individuals (50-65%).

Combination myocardial revascularization with MSC administration in patients with Acute Myocardial Infarction resulted in improvement of overall and local contractive myocardium functions and also normalization of systolic and diastolic filling of left ventricle.

What is claimed is:

a. A method for treating a coronary condition in a patient comprising:
i. providing a stem cell composition;
ii. administering the stem cell composition to the patient;
iii. wherein administering the stem cell composition treats the coronary condition.

b. The method of claim 1, wherein the stem cell composition is autologous, allogeneic, or a combination thereof with respect to the patient.

c. The method of claim 1, wherein the stem cell composition comprises mesenchymal stem cells.

d. The method of claim 3, wherein the mesenchymal stem cells are derived from cord blood, bone marrow, peripheral blood, adipose, or a combination thereof.

e. The method of claim 4, wherein the mesenchymal stem cells are derived from bone marrow.

f. The method of claim 3, wherein said mesenchymal stem cells are ischemic tolerant mesenchymal stem cells.

g. The method of claim 1, wherein the coronary condition is acute coronary syndrome.

h. The method of claim 1, wherein the stem cell composition is administered intravenously.
